(19) United States
(12) Patent Application Publication
(10) Pub. No.: US 2011/0313224 A1
(43) Pub. Date: Dec. 22, 2011

(54) PROCESS FOR THE PREPARATION OF AN OLEFINIC PRODUCT AND AN OXYGENATE CONVERSION CATALYST

(76) Inventors: Leslie Andrew Chewter, Amsterdam (NL); Ferry Winter, Amsterdam (NL); Jeroen Van Westrenen, Amsterdam (NL)

(21) Appl. No.: 13/140,865

(22) PCT Filed: Dec. 21, 2009
(86) PCT No.: PCT/EP09/67664

§ 371 Date: Aug. 18, 2011

(30) Foreign Application Priority Data
Dec. 22, 2008 (EP) 08172517.8

(51) Int. Cl. C07C 1/20; C01B 39/02

(52) U.S. Cl. 585/638; 423/700

(57) ABSTRACT

A process for the preparation of an olefinic product in the presence of a catalyst comprising an aluminosilicate having one-dimensional 10-membered ring channels and a silica-to-alumina-ratio of less than 170, and at least one Group IB metal at a metal loading of between 0.1 and 10 wt % of the aluminosilicate, the process comprising reacting an oxygenate feedstock under oxygenate conversion conditions to produce a reaction, product comprising ethylene and/or propylene. An oxygenate conversion catalyst is also claimed.

This invention relates to a process for the preparation of an olefinic product and an oxygenate conversion catalyst.

Processes for the preparation of olefins from oxygenates are known in the art. Of particular interest is often the production of light olefins, in particular ethylene and/or propylene. The oxygenate feedstock can for example comprise methanol and/or dimethylether, and an interesting route includes their production from synthesis gas derived from e.g. natural gas or via coal gasification.

For example, WO2007/135052 discloses a process wherein an alcohol and/or ether containing oxygenate feedstock and an olefinic co-feed are reacted in the presence of a zeolite having one-dimensional 10-membered ring channels to prepare an olefinic reaction mixture, and wherein part of the obtained olefinic reaction mixture is recycled as olefinic co-feed. With a methanol and/or dimethylether containing feedstock, and an olefinic co-feed comprising C4 and/or C5 olefins, an olefinic product rich in light olefins can be obtained.

By-products may be formed in the oxygenate to olefin reaction and such aromatics result in coking and deactivation of the catalyst. Accordingly the catalysts are normally reactivated periodically by removal of the coke deposits, normally by oxidation.

U.S. Pat. No. 4,845,063 discloses a cracking process using a zeolite having Group 1B metal cations, especially Ag, incorporated therein. Whilst largely teaching a process for cracking hydrocarbons, example 8, and column 8 line 21 to 30 also disclose the use of AgZSM-5 in a methanol to hydrocarbon reaction. It is apparent from consulting the references cited in column 8 of U.S. Pat. No. 4,845,063 that the products of this methanol to hydrocarbon synthesis are saturated, aromatic or C5+ hydrocarbons. A variety of zeolites are mentioned in column 3 lines 29 to 30 in connection with the invention described therein but these do not include zeolites having one-dimensional 10-membered ring channels. Whilst a broad range of silica-to-alumina ratios (SAR's) are disclosed, it is generally taught at the top of column 5 that zeolites with higher SAR showed a higher degree of improvement in hydrothermal stability than those with lower SAR.

U.S. Pat. No. 6,307,117 discloses a cracking process for cracking a hydrocarbon feedstock to obtain ethylene ethylene and propylene. The hydrocarbon feedstock contains C4+ hydrocarbons, and no or no more than impurities of oxygen-containing compounds. In all examples, a zeolite ZSM-5 based catalyst is used. ZSM-5 was treated with silver nitrate solution. Other zeolites are mentioned as well, but in any case the SAR of the zeolite used in the process disclosed is required to be in the range of from 200 to 5,000 and it is taught therein (column 8 lines 28-31) that if the SAR is less than 200 that the catalyst is likely to be deactivated due to coking during the cracking reaction.

During an oxygenate-to-olefins conversion reaction water is formed. Therefore the catalyst is exposed to hydrothermal conditions which can lead to aging effects that deteriorate its performance. There is a need for oxygenate conversion catalysts that exhibit improved hydrothermal stability.

According the present invention provides a process for the preparation of an olefinic product in the presence of a catalyst comprising an aluminosilicate having one-dimensional 10-membered ring channels and a silica-to-alumina-ratio of less than 170, and at least one Group IB metal at a metal loading of between 0.1 and 10 wt % of the aluminosilicate, the process comprising reacting an oxygenate feedstock under oxygenate conversion conditions to produce a reaction product comprising ethylene and/or propylene.

In another aspect the invention provides an oxygenate conversion catalyst, the catalyst comprising an aluminosilicate having one-dimensional 10-membered ring channels and a silica-to-alumina-ratio of less than 170, and silver at a silver loading of between 0.1 and 10 wt % of the aluminosilicate.

In process for the preparation of an olefinic product according to the present invention, there is provided an oxygenate conversion catalyst, the catalyst comprising an aluminosilicate having one-dimensional 10-membered ring channels and a silica-to-alumina ratio of less than 170, and at least one Group IB metal at a metal loading of between 0.1 and 10 wt % of the aluminosilicate.

The metal loading is based on the weight of the Group 1B metal in any form (e.g. atomic, ionic, or bound) on the total catalyst.

It has been found that aluminosilicates having one-dimensional 10-membered ring channels can be operated under oxygenate conversion conditions at high temperatures with acceptable deactivation due to coking. Moreover the detrimental coking reported in U.S. Pat. No. 6,307,117 at low SAR appears to be specific to the hydrocarbon cracking reaction, whereas it has now been found that a low SAR is preferred for oxygenate-to-olefin conversion reactions using aluminosilicates having one-dimensional 10-membered ring channels.

Group IB metals are also referred to as Group 11 metals. Group IB includes the elements copper, silver and gold. Silver is preferred.

The aluminosilicate is typically a molecular sieve. Preferably the loading is between 0.5 and 7.5 wt %, preferably between 1 and 5 wt %. Preferably the Group IB metal comprises silver.

Preferably the Group IB metal to aluminium ratio is in the range of 0.1-1.5, preferably 0.4-0.8.

The aluminosilicates having one-dimensional 10-membered ring channels according to the invention are understood to have only 10-membered ring channels in one direction which are not intersected by other 8, 10 or 12-membered ring channels from another direction.

Preferably, the aluminosilicate is selected from the group of TON-type (for example zeolite ZSM-22), MTT-type (for example zeolite ZSM-23), STF-type (for example SSZ-35), SFF-type (for example SSZ-44), EUO-type (for example ZSM-50), and EU-2-type aluminosilicates or mixtures thereof.

MTT-type catalysts are more particularly described in e.g. U.S. Pat. No. 4,076,842. For purposes of the present invention, MTT is considered to include its isotypes, e.g., ZSM-23, EU-13, ISI-4 and KZ-1.

TON-type aluminosilicates are more particularly described in e.g. U.S. Pat. No. 4,556,477. For purposes of the present invention, TON is considered to include its isotypes, e.g., ZSM-22, Theta-1, ISI-1, KZ-2 and NU-10.

EU-2-type aluminosilicates are more particularly described in e.g. U.S. Pat. No. 4,397,827. For purposes of the present invention, EU-2 is considered to include its isotypes, e.g., ZSM-48.

In a further preferred embodiment an aluminosilicate of the MTT-type, such as ZSM-23, and/or a TON-type, such as ZSM-22 is used.

The aluminosilicate is typically a molecular sieve. An aluminosilicate molecular sieve is also referred to as a zeolite. Molecular sieve and zeolite types are for example defined in Ch. Baerlocher and L. B. McCusker, Database of Zeolite Structures: http://www.iza-structure.org/databases/, which database was designed and implemented on behalf of the Structure Commission of the International Zeolite Association (IZA-SC), and based on the data of the 4th edition of the Atlas of Zeolite Structure Types (W. M. Meier, D. H. Olson and Ch. Baerlocher). The Atlas of Zeolite Framework Types, 5th revised edition 2001 and 6^{th} edition 2007 may also be consulted.

The present invention also provides a process for the manufacture of the catalyst of the invention, the process comprising:

(a) contacting a precursor catalyst comprising an aluminosilicate having one-dimensional 10-membered ring channels and a silica-to-alumina-ratio of less than 170 with a Group IB metal species, followed by (b) a heat-treatment at a temperature of above 250° C. in an atmosphere of inert gas and/or oxygen and/or steam;

such that the catalyst comprises a Group IB metal, at a metal loading of between 0.1 and 10 wt % of the aluminosilicate.

A precursor catalyst comprising aluminosilicate is understood to be a catalyst not containing Group IB metal at a metal loading of between 0.1 and 10 wt % of the aluminosilicate. The precursor catalyst can be aluminosilicate as such or in a formulation with other components such as a matrix, binder and/or filler component.

The contacting of step a) is suitably done by means of an ion exchange. A less preferred option is impregnation.

Step (b) is also referred to as a calcination.

When the aluminosilicates are prepared in the presence of organic cations, the aluminosilicate may be activated by heating in an inert or oxidative atmosphere to remove organic cations, for example, by heating at a temperature over 500° C. for 1 hour or more. The zeolite is typically obtained in the sodium or potassium form. The ammonium form can then be obtained by an ion exchange procedure with ammonium salts. The hydrogen form may then be obtained by another heat treatment, for example in an inert or oxidative atmosphere at a temperature over 300° C.

Preferably step (a) is performed when the aluminosilicate is in its ammonium form. This obviates the step of converting the ammonium-form aluminosilicate to a hydrogen form before the Group 1B metal exchange and so saves one calcination step to do this.

Preferably the Group IB metal species for contacting is a metal ion solution. For certain embodiments the metal ion solution is a silver nitrate solution and for such embodiments, preferably the molarity of the silver nitrate solution is from 0.01-2M, more preferably 0.04-0.5M, especially 0.1-0.2M.

The aluminosilicate having one-dimensional 10-membered ring channels has a silica-to-alumina ratio (SAR) in the range of from 1 to 170, preferably in the range of from 20 to 150. The SAR is defined as the molar ratio of SiO₂/Al₂O₃ corresponding to the composition of the aluminosilicate.

For ZSM-22, a SAR in the range of 40-150 is preferred, in particular in the range of 70-120. Good performance in terms of activity and selectivity has been observed with a SAR of about 100.

For ZSM-23, an SAR in the range of 20-120 is preferred, in particular in the range of 30-80. Good performance in terms of activity and selectivity has been observed with a SAR of about 50.

In a special embodiment the reaction is performed in the presence of a more-dimensional molecular sieve, such as ZSM-5. Suitably to this end the oxygenate conversion catalyst comprises at least 1 wt %, based on total molecular sieve in the oxygenate conversion catalyst, of a further molecular sieve having more-dimensional channels, in particular at least 5 wt %, more in particular at least 8 wt %.

The further molecular sieve having more-dimensional channels is understood to have intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably the channels in at least one of the directions are 10-membered ring channels. The further molecular sieve can be for example a FER type zeolite which is a two-dimensional structure and has 8- and 10-membered rings intersecting each other. Preferably however the intersecting channels in the further molecular sieve are each 10-membered ring channels. Thus the further molecular sieve may be a zeolite, or a SAPO-type (silicoaluminophosphate) molecular sieve. More preferably however the further molecular sieve is a zeolite. A preferred further molecular sieve is an MFI-type zeolite, in particular zeolite ZSM-5.

The presence of the further molecular sieve in the oxygenate conversion catalyst was found to improve stability (slower deactivation during extended runs) and hydrothermal stability compared to a catalyst with only the one-dimensional molecular sieve and without the more-dimensional molecular sieve. Without wishing to be bound by a particular hypothesis or theory, it is presently believed that this is due to the possibility for converting larger molecules by the further molecular sieve having more-dimensional channels, that were produced by the first molecular sieve having one-dimensional 10-membered ring channels, and which would otherwise form coke. When the one-dimensional aluminosilicate and the more-dimensional molecular sieve are formulated such that they are present in the same catalyst particle, such as in a spray-dried particle, this intimate mix was found to improve the selectivity towards ethylene and propylene, more in particular towards ethylene.

The weight ratio between the aluminosilicate having one-dimensional 10-membered ring channels, and the further molecular sieve having more-dimensional channels can be in the range of from 1:100 to 100:1. Preferably the further molecular sieve is the minority component, i.e. the above weight ratio is 1:1 to 100:1, more preferably in the range of 9:1 to 2:1.

Preferably the further molecular sieve is an MFI-type aluminosilicate, in particular zeolite ZSM-5, having a silica-to-alumina ratio (SAR) of at least 60, more preferably at least 80, even more preferably at least 100, yet more preferably at least 150. At higher SAR the percentage of C4 saturates in the C4 totals produced is minimized. In special embodiments the oxygenate conversion catalyst can comprise less than 35 wt % of the further molecular sieve, based on the total molecular sieve in the oxygenate conversion catalyst, in particular less than 20 wt %, more in particular less than 18 wt %, still more in particular less than 15 wt %.

Preferably the further molecular sieve is in the sodium, hydrogen or ammonium form. Preferably therefore the further molecular sieve is not silver exchanged.

However if the aluminosilicate with one-dimensional 10-membered ring channels and the further molecular sieve are treated with the silver nitrate solution after their combination, then both will be silver exchanged.

In one embodiment the oxygenate conversion catalyst can comprise more than 50 wt %, at least 65 wt %, based on total molecular sieve in the oxygenate conversion catalyst, of the aluminosilicate having one-dimensional 10-membered ring channels. The presence of a majority of such aluminosilicate strongly determines the predominant reaction pathway.

The aluminosilicate can be used as such or in a formulation, such as in a mixture or combination with a so-called binder material and/or a filler material, and optionally also with an active matrix component. Other components can also be present in the formulation. If one or more aluminosilicates are used as such, in particular when no binder, filler, or active matrix material is used, the aluminosilicate itself is/are referred to as oxygenate conversion catalyst. In a formulation, the aluminosilicate in combination with the other components of the mixture such as binder and/or filler material is/are referred to as oxygenate conversion catalyst.

It is desirable to provide a catalyst having good mechanical or crush strength, because in an industrial environment the catalyst is often subjected to rough handling, which tends to break down the catalyst into powder-like material. The latter causes problems in the processing. Preferably the aluminosilicate is therefore incorporated in a binder material. Examples of suitable materials in a formulation include active and inert materials and synthetic or naturally occurring zeolites as well as inorganic materials such as clays, silica, alumina, silica-alumina, titania, zirconia and aluminosilicate. For present purposes, inert materials, such as silica, are preferred because they may prevent unwanted side reactions which may take place in case a more acidic material, such as alumina or silica-alumina is used.

In the process for the preparation of an olefinic product in the presence of a catalyst as defined herein, the process comprising reacting an oxygenate feedstock in oxygenate conversion conditions to produce a reaction product comprising ethylene and/or propylene. The invention thus provides the advantageous use of the catalyst of the present invention in an oxygenate-to-olefins conversion reaction.

Thus the process according to the invention is designed to produce lower olefins for recovery and onward processing and/or sale. Typically therefore, a stream comprising at least 50 wt %, preferably at least 75 wt %, C2 to C3 olefins (ethylene and/or propylene) is separated from the reaction product, based on total reaction product.

To separate the streams, the olefinic product is typically fractionated.

The skilled artisan knows how to separate a mixture of hydrocarbons into various fractions, and how to work up fractions further for desired properties and composition for further use. The separations can be carried out by any method known to the skilled person in the art to be suitable for this purpose, for example by vapour-liquid separation (e.g. flashing), distillation, extraction, membrane separation or a combination of such methods. Preferably the separations are carried out by means of distillation. It is within the skill of the artisan to determine the correct conditions in a fractionation column to arrive at such a separation. He may choose the correct conditions based on, inter alia, fractionation temperature, pressure, trays, reflux and reboiler ratios.

Indeed preferably the reaction product is separated, typically fractionated, into at least a light olefinic fraction comprising at least 50%, preferably at least 90% ethylene, and a heavier olefinic fraction comprising C4 olefins and less than 10 wt % of C5+ hydrocarbon species.

Preferably also a water-rich fraction is obtained. Also a lighter fraction comprising methane, carbon monoxide, and/or carbon dioxide can be obtained, as well as one or more heavy fractions comprising C5+ hydrocarbons. Such heavy fraction can for example be used as gasoline blending component.

In the process also a significant amount of propylene is normally produced. The propylene can form part of the light olefinic fraction comprising ethene, and which can suitably be further fractionated into various product components. Propylene can also form part of the heavier olefinic fraction comprising C4 olefins. The various fractions and streams referred to herein can be obtained by fractionating in various stages, and also by blending streams obtained during the fractionation. Typically, an ethylene and a propylene stream of predetermined purity such as pipeline grade, polymer grade, chemical grade or export quality will be obtained from the process, and also a stream rich in C4 comprising C4 olefins and optionally C4 paraffins.

It shall be clear that a recycle stream, can be composed from quantities of various fractionation streams. So, for example, some amount of a propylene-rich stream can be blended into a C4 olefin-rich stream. In a particular embodiment at least 90 wt % of the heavier olefinic fraction comprising C4 olefins can be formed by the overhead stream from a debutaniser column receiving the bottom stream from a depropanizer column at their inlet, more in particular at least 99 wt % or substantially all.

Suitably the olefinic reaction effluent comprises less than 10 wt %, preferably less than 5 wt %, more preferably less than 1 wt %, of C6-C8 aromatics. Producing low amounts of aromatics is desired since any production of aromatics consumes oxygenate which is therefore not converted to lower olefins.

The oxygenate feedstock suitably comprises oxygenate species having an oxygen-bonded methyl group, such as methanol or dimethylether. Preferably the oxygenate feedstock comprises at least 50 wt % of methanol and/or dimethylether, more preferably at least 80 wt %, most preferably at least 90 wt %.

The oxygenate feedstock can be obtained from a different or separate reactor, which converts methanol at least partially into dimethylether. In this way, water may be removed by distillation and so less water is present in the process of converting oxygenate to olefins, which has advantages for the process design and lowers the severity of hydrothermal conditions the catalyst is exposed to.

The oxygenate feedstock can comprise an amount of water, preferably less than 10 wt %, more preferably less than 5 wt %. Preferably the oxygenate feedstock contains essentially no hydrocarbons other than oxygenates, i.e. less than 5 wt %, preferably less than 1 wt %.

In one embodiment, the oxygenate is obtained as a reaction product of synthesis gas. Synthesis gas can for example be generated from fossil fuels, such as from natural gas or oil, or from the gasification of coal. Suitable processes for this purpose are for example discussed in Industrial Organic Chemistry, Klaus Weissermehl and Hans-Jürgen Arpe, 3rd edition, Wiley, 1997, pages 13-28. This book also describes the manufacture of methanol from synthesis gas on pages 28-30.

In another embodiment the oxygenate is obtained from biomaterials, such as through fermentation. For example by a process as described in DE-A-10043644.

Preferably the oxygenate feedstock is reacted to produce the olefinic product in the presence of an olefinic co-feed. By an olefinic composition or stream, such as a reaction product comprising, an olefinic product, product fraction, fraction, effluent, reaction effluent or the like is understood a composition or stream comprising one or more olefins, unless specifically indicated otherwise. Other species can be present as well. Apart from olefins, the olefinic co-feed may contain other hydrocarbon compounds, such as for example paraffinic compounds. Preferably the olefinic co-feed comprises an olefinic portion of more than 50 wt %, more preferably more than 60 wt %, still more preferably more than 70 wt %, which olefinic portion consists of olefin(s). The olefinic co-feed can also consist essentially of olefin(s).

Any non-olefinic compounds in the olefinic co-feed are preferably paraffinic compounds. Such paraffinic compounds are preferably present in an amount in the range of from 0 to 50 wt %, more preferably in the range of from 0 to 40 wt %, still more preferably in the range of from 0 to 30 wt %.

By an olefin is understood an organic compound containing at least two carbon atoms connected by a double bond. The olefin can be a mono-olefin, having one double bond, or a poly-olefin, having two or more double bonds. Preferably olefins present in the olefinic co-feed are mono-olefins. C4 olefins, also referred to as butenes (1-butene, 2-butene, iso-butene, and/or butadiene), in particular C4 mono-olefins, are preferred components in the olefinic co-feed.

Preferably the olefinic co-feed is at least partially obtained by a recycle stream formed by recycling a suitable fraction of the reaction product comprising C4 olefin.

In one embodiment at least 70 wt % of the olefinic co-feed, during normal operation, is formed by the recycle stream, preferably at least 90 wt %, more preferably at least 99 wt %. Most preferably the olefinic co-feed is during normal operation formed by the recycle stream, so that the process converts oxygenate feedstock to predominantly light olefins without the need for an external olefins stream. During normal operation means for example in the course of a continuous operation of the process, for at least 70% of the time on stream. The olefinic co-feed may need to be obtained from an external source, such as from a catalytic cracking unit or from a naphtha cracker, during start-up of the process, when the reaction effluent comprises no or insufficient C4+ olefins.

A particularly preferred olefinic recycle stream is a C4 fraction containing C4 olefin(s), but which can also contain a significant amount of other C4 hydrocarbon species, in particular C4 paraffins, because it is difficult to economically separate C4 olefins and paraffins, such as by distillation.

In a preferred embodiment the olefinic co-feed and preferably also the recycle stream comprises C4 olefins and less than 10 wt % of C5+ hydrocarbon species, more preferably at least 50 wt % of C4 olefins, and at least a total of 70 wt % of C4 hydrocarbon species.

The olefinic co-feed and preferably also the recycle stream, can in particular contain at least a total of 90 wt % of C4 hydrocarbon species. In a preferred embodiment, the olefinic co-feed comprises less than 5 wt % of C5+ olefins, preferably less than 2 wt % of C5+ olefins, even more preferably less than 1 wt % of C5+ olefins, and likewise the recycle stream. In another preferred embodiment, the olefinic co-feed, comprises less than 5 wt % of C5+ hydrocarbon species, preferably less than 2 wt % of C5+ hydrocarbon species even more preferably less than 1 wt % of C5+ hydrocarbon species, and likewise the recycle stream.

Thus in certain preferred embodiments, the olefinic portion of the olefinic co-feed, and of the recycle stream, comprises at least 90 wt % of C4 olefins, more preferably at least 99 wt %. Butenes as co-feed have been found to be particularly beneficial for high ethylene selectivity. Therefore one particularly suitable recycle stream consists essentially, i.e. for at least 99 wt %, of 1-butene, 2-butene (cis and trans), isobutene, n-butane, isobutane, butadiene.

In certain embodiments, the recycle stream can also comprise propylene. This may be preferred when a particularly high production of ethylene is desired, so that part or all of the propylene produced, such as at least 5 wt % thereof, is recycled together with C4 olefins.

The preferred molar ratio of oxygenate in the oxygenate feedstock to olefin in the olefinic co-feed depends on the specific oxygenate used and the number of reactive oxygen-bonded alkyl groups therein. Preferably the molar ratio of oxygenate to olefin in the total feed lies in the range of 10:1 to 1:10, more preferably in the range of 5:1 to 1:5 and still more preferably in the range of 3:1 to 1:3.

In a preferred embodiment wherein the oxygenate comprises only one oxygen-bonded methyl group, such as methanol, the molar ratio preferably lies in the range of from 5:1 to 1:5 and more preferably in the range of 2.5:1 to 1:2.5.

In another preferred embodiment wherein the oxygenate comprises two oxygen-bonded methyl groups, such as for example dimethylether, the molar ratio preferably lies in the range of from 5:2 to 1:10 and more preferably in the range of 2:1 to 1:4. Most preferably the molar ratio in such a case is in the range of 1.5:1 to 1:3.

The process of the present invention can be carried out in a batch, continuous, semi-batch or semi-continuous manner. Preferably the process of the present invention is carried out in a continuous manner.

If the process is carried out in a continuous manner, the process may be started up by using olefins obtained from an external source for the olefinic co-feed, if used. Such olefins may for example be obtained from a steam cracker, a catalytic cracker, alkane dehydrogenation (e.g. propane or butane dehydrogenation). Further, such olefins can be bought from the market.

When a molecular sieve having more-dimensional channels such as ZSM-5 is present in the oxygenate conversion catalyst, even in minority compared to the aluminosilicate having one-dimensional 10-membered ring channels, start up is possible without an olefinic co-feed from an external source. ZSM-5 for example is able to convert an oxygenate to an olefin-containing product, so that a recycle can be established.

Typically the oxygenate conversion catalyst deactivates in the course of the process although the presence of the Group IB metal according to the invention mitigates this deactivation. Conventional catalyst regeneration techniques can be employed, such as oxidation of coke in a regenerator. The aluminosilicate having one-dimensional 10 membered ring channels used in the process of the present invention can have any shape known to the skilled person to be suitable for this purpose, for it can be present in the form of spray-dried particles, spheres, tablets, rings, extrudates, etc. Extruded catalysts can be applied in various shapes, such as, cylinders and trilobes.

The reactor system used to produce the olefins may be any reactor known to the skilled person and may for example contain a fixed bed, moving bed, fluidized bed, riser reactor and the like. A riser reactor system is preferred, and in a particular embodiment a riser reactor system comprising a plurality of serially arranged riser reactor stages is used.

The reaction to produce the olefins can be carried out over a wide range of temperatures and pressures. Suitably, however, the oxygenate feed and olefinic co-feed are contacted with the aluminosilicate at a temperature in the range of from 200° C. to 650° C. In a further preferred embodiment the temperature is in the range of from 250° C. to 600° C., more preferably in the range of from 300° C. to 550° C., most preferably in the range of from 450° C. to 550° C. Preferably the reaction to produce the reaction product comprising the C2-C4 olefins is conducted at a temperature of more than 450° C., preferably at a temperature of 460° C. or higher, more preferably at a temperature of 490° C. or higher. At higher temperatures a higher activity and ethylene selectivity is observed. Temperatures referred to hereinabove represent reaction temperatures, and it will be understood that a reaction temperature can be an average of temperatures of various feed streams and the catalyst in the reaction zone.

For certain embodiments the catalyst is contacted with a gas comprising steam, before reacting the oxygenate feedstock to produce the reaction product. This is in marked contrast to the normal teaching of catalyst preparation and usage for oxygenate to hydrocarbon conversion, where hydrothermal conditions are known to cause deactivation of the catalyst. However, in the present process, especially for embodiments where the catalyst comprises a TON-type (for example zeolite ZSM-22), such a pre-treatment may be undertaken which increases the catalytic activity of the catalyst.

Thus optionally the catalyst may pre-treated by contact with a gas comprising steam before the process for oxygenate to olefin process starts. The steam is normally at a temperature of over 100° C., preferably in the range of from 350 to 750° C., preferably of from 375to 675°. The gas comprising steam can suitably be 100% steam, but steam diluted in an inert gas can also be used

Moreover, a diluent may be fed into the reactor system at the same time as the oxygenate, and the olefinic co-feed, if used. It is preferred to operate without a diluent or with a minimum amount of diluent, such as less than 200 wt % of diluent based on the total amount of oxygenate feed, in particular less than 100 wt %, more in particular less than 20 wt %. Any diluent known by the skilled person to be suitable for such purpose can be used. Such diluent can for example be a paraffinic compound or mixture of compounds. Preferably, however, the diluent is an inert gas. The diluent can be argon, nitrogen, and/or steam. Of these, steam is the most preferred diluent. For example, the oxygenate feed and optionally olefinic co-feed can be diluted with steam, for example in the range of from 0.01 to 10 kg steam per kg oxygenate feed. In one embodiment small amounts of water are added in order to improve the stability of the catalyst by reducing coke formation.

Embodiments of the invention will now be described, by way of example only.

Various catalyst samples were prepared and treated with a silver solution in order to effect silver exchange. Their activity and selectivity was compared with equivalent catalyst samples which did not undergo silver exchange.

To prepare the various samples, zeolite powder was pressed into tablets and the tablets were broken into pieces and sieved. For the subsequent catalytic testing, the sieve fraction of 40-60 mesh was used.

A first series of catalyst samples were prepared comprising ZSM-23.

### Catalyst Sample 1

ZSM-23 with a silica-to-alumina ratio (SAR) of 46 in the ammonium form was ion-exchanged with silver. To this end, 20 g of the ZSM-23 was added to 200 ml of an aqueous solution containing 0.2 mol·l⁻¹ silver nitrate in demineralised water, kept at 80° C. for 2 hours whilst being stirred vigorously. Afterwards, the material was filtered off, and dried at 120° C. The ion-exchanged ZSM-23 was then calcined at 550° C.

The resulting catalyst sample contained 3.5 wt % of silver and the Ag to Al ratio was 0.52 (mol:mol).

### Catalyst Sample 2

Catalyst sample 2 was prepared by aging catalyst sample 1 at 600° C. for 5 hours in a gaseous stream containing 30 vol % steam and 70 vol % Ar.

### Catalyst Sample 3

Catalyst sample 3 was prepared by aging catalyst sample 1 at 600° C. for 14 hours in a gaseous stream containing 30 vol % steam and 70 vol % Ar.

### Catalyst Sample 4

20 grams of ZSM-23 with a SAR of 46 was added into 200 ml of an aqueous solution containing 0.2 mol·l⁻¹ silver nitrate. The resulting suspension was kept at 80° C. and was vigorously stirred for 2 hours. Afterwards, the material was filtered off and dried at 120° C. The procedure was repeated two times after which the material was calcined at 550° C.

The resulting catalyst contained 4.5 wt % of silver and the Ag to Al ratio was 0.69 (mol:mol).

### Catalyst Sample 5

Catalyst Sample 5 was prepared by aging catalyst sample 4 at 600° C. for 14 hours in a gaseous stream containing 30 vol % steam and 70 vol % Ar.

Catalyst Sample 6 is a comparative example wherein ZSM-23 with SAR 46 and without a group 1B metal is used.
Catalyst Sample 7 is another comparative example where catalyst sample 6 was treated at 600° C. for 5 hours in a gaseous stream containing 30 vol % steam and 70 vol % Ar.
Catalyst Sample 8 is another comparative example where catalyst sample 6 was treated at 600° C. for 14 hours in a gaseous stream containing 30 vol % steam and 70 vol % Ar.

The relative activity determined by catalytic testing of catalyst samples 1 to 8 is shown in Table 1 below. The catalytic testing reaction was performed using a quartz reactor tube of 3.6 mm internal diameter. The catalyst samples were heated in Argon to the reaction temperature. The gaseous feed consisting of 3 vol % dimethyl ether, 3 vol % 1-butene, 2 vol % steam balanced in N₂ was passed over the catalyst at atmospheric pressure (1 bar). Gas hourly space velocity (GHSV) is based on total gas flow (ml·g_{cat}⁻¹·h⁻¹).

TABLE 1
Relative activity (ZSM-23 containing catalysts)

| | Aging | Activity compared |
|---|---|---|
| | time | to unmodified |
| | (h) | ZSM-23 SAR 46 |
| Catalyst sample 1 | 0 | 85 |
| Catalyst sample 2 | 5 | 55 |
| Catalyst sample 3 | 14 | 35 |
| Catalyst sample 4 | 0 | 80 |
| Catalyst sample 5 | 14 | 35 |
| Catalyst sample 6 | 0 | 100 |
| (comparative) | | |
| Catalyst sample 7 | 5 | 25-30 |
| (comparative) | | |
| Catalyst sample 8 | 14 | 5-10 |
| (comparative) | | |

As shown in Table 1 the activity of the comparative catalyst samples deteriorates after aging, with catalyst sample 8 aged for 14 hours having an activity of only around 5-10% of its original activity, catalyst sample 6.

Catalyst sample 1 having a metal loading of 3.5 wt % silver does initially start with lower activity compared to the activity of the untreated catalyst sample (catalyst sample 6). However the deterioration following catalyst aging is much less compared with the untreated sample—catalyst sample 2 and 3 have much better activity compared to catalysts 7 and 8. Catalyst sample 5 having 4.5% Ag also shows improved activity compared to comparative catalyst sample 8 when both have been aged for 14 hours.

A second series of catalyst samples were prepared comprising ZSM-22 zeolite.

### Catalyst Sample 9

ZSM-22 with a SAR of 107 was suspended in 0.1 mol·l⁻¹ silver nitrate solution. The resulting suspension was stirred vigorously at 80° C. for 2 hours. The material was filtered off and dried at 120° C. The catalyst was calcined at 550° C. for 2 h. The resulting catalyst contained 1.4 wt % of silver and the Ag to Al ratio was 0.45 (mol:mol).

### Catalyst Sample 10

Catalyst sample 10 was prepared by aging catalyst sample 9 at 600° C. for 5 hours in a gaseous stream containing 30 vol % steam and 70 vol % Ar.

### Catalyst Sample 11

Catalyst sample 10 was prepared by aging catalyst sample 9 at 600° C. for 14 hours in a gaseous stream containing 30 vol % steam and 70 vol % Ar.

Catalyst Sample 12 is a comparative example and comprises only ZSM-22 with SAR 107 and no silver.
Catalyst Sample 13 is another comparative example where catalyst sample 12 was aged by treatment at 600° C. for 5 hours in a gaseous stream containing 30 vol % steam and 70 vol % Ar.
Catalyst Sample 14 is another comparative example where catalyst sample 12 was aged by treatment at 600° C. for 14 hours in a gaseous stream containing 30 vol % steam and 70vol % Ar.
Catalyst Sample 15 is a comparative example wherein only ZSM-22 with SAR 281 without a group 1B metal is used.

The comparative catalyst sample 12 comprising ZSM-22 has an activity of around 80-85% of that of the comparative catalyst sample 1 comprising ZSM-23. The relative activity of the catalyst samples 9 to 14 comprising ZSM-22 are detailed in Table 2 below.

TABLE 2
Relative activity (ZSM-22 containing catalysts)

| | Aging | Activity compared |
|---|---|---|
| | time | to unmodified |
| | (h) | ZSM-22 SAR 107 |
| Catalyst sample 9 | 0 | 30 |
| Catalyst sample 10 | 5 | 100 |
| Catalyst sample 11 | 14 | 90-95 |
| Catalyst sample 12 | 0 | 100 |
| (comparative) | | |
| Catalyst sample 13 | 5 | 70 |
| (comparative) | | |
| Catalyst sample 14 | 14 | 30 |
| (comparative) | | |

The comparative samples (12 to 14) show the expected deterioration after aging. Catalyst sample 9 comprising 1.4% silver initially starts with a lower activity but a small degree of aging surprisingly increases its activity. After 14 hours the activity of the silver-exchanged catalyst 11 is significantly better (90-95%) compared to the sample without silver (catalyst sample 14) which has also been aged for the same period of time.

The results in Tables 1 and 2 show the improved activity after aging of catalyst samples which have undergone a silver exchange step. Surprisingly the silver exchanged ZSM-22, catalyst sample 10, showed increased activity after a moderate amount of aging (5 hours). Certain embodiments of the invention, especially those comprising silver exchanged ZSM-22 may include an aging step, such as treatment with steam, before performing the oxygenate to olefin process.

The effluent from the reactor was analyzed by gas chromatography (GC) to determine the product composition which is shown in Table 3 below.

TABLE 3
Feed: 3 vol % 1-butene, 3 vol % dimethyl ether. Temperature (° C.) 525. GHSV (ml · g_{cat}⁻¹ · h⁻¹), 15,000

| | Catalyst | | | | | | | | | | | | | | |
|---|---|---|---|---|---|---|---|---|---|---|---|---|---|---|---|
| | Catalyst sample | | | Catalyst sample | | | Catalyst | | | Catalyst sample | | | Catalyst sample | | |
| | 12 (comparative) | | | 14 (comparative) | | | sample 9 | | | 11 | | | 15 (comparative) | | |
| | Sample Content | | | | | | | | | | | | | | |
| | ZSM-22 | | | ZSM-22HT14 | | | Ag-ZSM-22 | | | Ag-ZSM-22 | | | ZSM-22 | | |
| | (SAR 107) | | | (SAR 107) | | | (SAR 107) | | | (SAR 107) | | | SAR 281 | | |
| | Aging time (h) | | | | | | | | | | | | | | |
| | 0 | | | 14 | | | 0 | | | 14 | | | 0 | | |
| | Time on stream (h) | | | | | | | | | | | | | | |
| | 1 | 8 | 17 | 1 | 8 | 17 | 1 | 8 | 16 | 1 | 8 | 16 | 1 | 8 | 17 |
| DME | 100 | 100 | 100 | 100 | 100 | 97.3 | 100 | 100 | 100 | 100 | 100 | 100 | 100 | 89.8 | 70.4 |
| conversion | | | | | | | | | | | | | | | |
| (%) | | | | | | | | | | | | | | | |
| Methane | 0.4 | 0.4 | 0.4 | 0.3 | 0.3 | 0.3 | 0.5 | 0.4 | 0.5 | 0.3 | 0.3 | 0.3 | 0.3 | 0.3 | 0.2 |
| (wt %) | | | | | | | | | | | | | | | |
| Ethylene | 17.2 | 14.1 | 9.8 | 5.5 | 2.6 | 1.0 | 7.4 | 5.1 | 3.7 | 17.9 | 9.7 | 4.6 | 9.8 | 0.7 | 0.3 |
| (wt %) | | | | | | | | | | | | | | | |
| Propylene | 47.7 | 50.4 | 46.5 | 35.8 | 26.4 | 11.2 | 41.2 | 41.1 | 36.9 | 49.9 | 45.7 | 37.2 | 38.4 | 7.0 | 2.5 |
| (wt %) | | | | | | | | | | | | | | | |
| C4 | 26.1 | 21.9 | 20.9 | 23.1 | 20.9 | 15.9 | 21.0 | 20.2 | 19.6 | 23.7 | 21.9 | 21.6 | 27.4 | 13.1 | 16.5 |
| isomers | | | | | | | | | | | | | | | |
| (wt %) | | | | | | | | | | | | | | | |
| C5 | 4.6 | 9.6 | 18.1 | 31.6 | 37.6 | 37.8 | 21.5 | 29.3 | 32.6 | 4.7 | 19.2 | 30.6 | 21.8 | 40.1 | 40.4 |
| isomers | | | | | | | | | | | | | | | |
| (wt %) | | | | | | | | | | | | | | | |
| C6-C9* | 3.5 | 3.1 | 3.8 | 3.7 | 12.2 | 26.9 | 4.8 | 3.6 | 6.6 | 3.0 | 3.0 | 5.5 | 2.1 | 29.9 | 19.6 |
| isomers | | | | | | | | | | | | | | | |
| (wt %) | | | | | | | | | | | | | | | |
| C6-C8 | 0.4 | 0.4 | 0.4 | 0.1 | 0 | 0.1 | 3.6 | 0.2 | 0.2 | 0.4 | 0.2 | 0.1 | 0.1 | 0 | 0 |
| aromatics | | | | | | | | | | | | | | | |
| (wt %) | | | | | | | | | | | | | | | |
| Ethylene/ | 0.36 | 0.28 | 0.21 | 0.15 | 0.10 | 0.09 | 0.18 | 0.13 | 0.10 | 0.36 | 0.21 | 0.12 | 0.26 | 0.10 | 0.11 |
| propylene | | | | | | | | | | | | | | | |
| ratio (wt/wt) | | | | | | | | | | | | | | | |

The composition (product yields in wt %) shown in Table 3 has been calculated on a weight basis of all hydrocarbons analyzed.

In Table 3 the product distribution is given for selected experiments with Ag-ZSM-22 and ZSM-22 both having a SAR of 107 and the comparative catalyst sample 15 with ZSM-22 having a SAR of 281.

This untreated catalyst sample 15 shows inferior performance compared to the untreated ZSM-22 having a SAR of 107 (catalyst sample 12). Thus the silver exchange was conducted on catalyst samples (9 and 11) which also have a SAR of 107, that is, the same SAR as that of untreated catalyst sample 12.

The comparative catalyst samples 12 and 14 show an expected result—that the more aged catalyst sample 14 has generally a poorer performance than catalyst sample 12, for example, it can be seen from Table 3 that the amount of ethylene and propylene is less.

Considering catalyst sample 9 which has undergone silver treatment but no aging, the results in Table 3 show that it (initially) results in less ethylene compared to comparative catalyst sample 12 which has no silver exchange. As noted from the results in Table 2, the initial performance of the ZSM-22 without aging is poorer than that of the comparative example. However, when comparing the aged silver exchanged catalyst, catalyst sample 11, compared to the equivalent catalyst sample with no silver exchange, catalyst sample 14, the silver-exchanged catalyst sample has much better performance—a much improved amount of ethylene compared to the untreated sample is apparent from table 3.

Thus embodiments of the present invention, such as those detailed above having undergone silver exchange, show a clearly improved performance for oxygenate to olefin conversion reactions.

1. A process for the preparation of an olefinic product in the presence of a catalyst comprising an aluminosilicate having one-dimensional 10-membered ring channels and a silica-to-alumina-ratio of less than 170, and at least one Group IB metal at a metal loading of between 0.1 and 10 wt % of the aluminosilicate, the process comprising reacting an oxygenate feedstock under oxygenate conversion conditions to produce a reaction product comprising ethylene and/or propylene.

2. A process as claimed in claim 1, wherein the metal loading of the catalyst is between 0.5 and 7.5 wt % of the aluminosilicate.

3. A process as claimed in claim 1, wherein the Group IB metal of the catalyst comprises silver.

4. A process as claimed in claim 1, wherein the aluminosilicate of the catalyst comprises a TON-type aluminosilicate.

5. A process as claimed in claim 4, wherein the TON-type aluminosilicate comprises ZSM-22 which has been exposed to a gas comprising steam, at a temperature of at least 100° C. before use.

6. A process as claimed in claim 1, wherein the aluminosilicate having one-dimensional 10-membered ring channels of the catalyst has a silica-to-alumina ratio in the range of from 20 to 150.

7. A process as claimed in claim 1, wherein the aluminosilicate of the catalyst comprises an MTT-type aluminosilicate.

8. A process as claimed in claim 1, wherein the catalyst further comprises a molecular sieve having more-dimensional channels.

9. A process as claimed in claim 1, wherein a stream comprising at least 50 wt %, C2 to C3 olefins is separated from the reaction product, based on total reaction product.

10. A process as claimed in claim 1, wherein the catalyst is contacted with a gas comprising steam, at a temperature of at least 100° C., before reacting the oxygenate feedstock to produce the reaction product.

11. A process as claimed in claim 1, wherein the oxygenate feedstock is reacted to produce the reaction product in the presence of an olefinic co-feed.

12. A process as claimed in claim 1, wherein the catalyst further comprises a molecular sieve having more-dimensional channels.

13. An oxygenate conversion catalyst, the catalyst comprising an aluminosilicate having one-dimensional 10-membered ring channels and a silica-to-alumina-ratio of less than 170, and silver at a silver loading of between 0.1 and 10 wt % of the aluminosilicate.

14. An oxygenate conversion catalyst as claimed in claim 13, wherein the catalyst further comprises a molecular sieve having more-dimensional channels.
